(19) United States
(12) Patent Application Publication
(10) Pub. No.: US 2005/0281313 A1
(43) Pub. Date: Dec. 22, 2005

(54) Method for measuring the temperature in the body of human or animal with acoustic inversion

(76) Inventors: Zuwen Qian, Beijing (CN); Liulin Xiong, Beijing (CN); Jinsheng Yu, Beijing (CN); Houqing Zhu, Beijing (CN); Daoyuan Shao, Beijing (CN); Xiaodong Wu, Beijing (CN)

Correspondence Address: BANNER & WITCOFF, 1001 G STREET N W, SUITE 1100, WASHINGTON, DC, 20001, US

(21) Appl. No.: 11/140,489

(22) Filed: May 31, 2005

(30) Foreign Application Priority Data
Jun. 4, 2004 (CN) 200410046091.9

(51) Int. Cl. G01K 11/22; A61B 8/00

(52) U.S. Cl. 374/117; 600/438; 600/439; 702/131

(57) ABSTRACT

A method for measuring the local temperature in the body of human or animal is provided. In the method, a first ultrasonic wave is transmitted to a region to be measured, which has temperature T, under the guiding of M-type ultrasound. The reflected ultrasonic wave from a particular reflection surface is received to obtain a first parameter. Then the temperature of the region to be measured is modified to T+ΔT. A second ultrasonic wave is transmitted to the region to be measured. The reflected ultrasonic wave from the second ultrasonic wave reflected by a particular reflection surface is received to obtain a second parameter. A ratio of the measured value of 2^{nd} parameter to that of 1^{st} parameter can be obtained. On the other hand, a theoretical ratio of the second parameter to the first parameter can also be obtained through theoretical calculation. The objection function which involves the differences between the theoretical ratio and measured ratio can be minimized by an optimization method. The local temperature increment ΔT of the region to be measured will be obtained with an inversion method.

This application claims priority from Chinese Patent Application No. 200410046091.9 filed on Jun. 4, 2004, which is incorporated herein by reference.

### TECHNICAL FIELD

This invention relates to a non-invasive method for measuring the temperature in the body of human or animal. Specifically, it relates to the application of High Intensive Focused Ultrasound (HIFU) to generate heat with high temperature in the body of human (animal) to react on pathological changes in treated regions. In order to measure the temperature in treated regions, the present invention provides a non-invasive measuring method utilizing acoustic inversion and provides an apparatus using the method.

### BACKGROUND ART

Nowadays, the focused ultrasonic therapeutic apparatus is one of the hotspots in the medical study in view of its good results in the clinical application. The HIFU generates high heat in the human (animal) body to cure lesions in the treated region. If the temperature is not high enough, the curative effects will not be perfect because the tumor cells cannot be destroyed. If the temperature is too high, malpractice will be made with burns of human body. Typically, there are two methods for the measurement of the temperature: invasive method or non-invasive method. The former one is to insert a thermometer into human body to measure the temperature. It is difficult to be utilized in practical treatment because of its invasive nature and hurts to patients. The latter method is to perform non-invasive measurement outside of the human body. It will not cause the above trouble if realized. But as far as we know, there still has no effective method to measure (clinically) the temperature in the treated region.

In fact, the doctors normally determine the parameters for the treatment according to their own clinical experience. Therefore, the best results cannot be ensured with the randomness on the parameters. Some non-invasive temperature measuring methods have been proposed, for example, Chinese granted Patent No. CN1358549A has suggested a predication method for the temperature at the focus of HIFU thermal therapeutic apparatus. It makes theoretical derivation on the acoustic field intensity distribution and temperature field distribution from the wave source. The predicted value of the temperature at the focus can be calculated with treatment parameter settings, such as input electrical power, conversion efficiency of transmitter, tissue characteristics and wave source characteristics, etc. CN1358549A forms a look-up table for temperature through calculating the theoretical focus temperature under different conditions, corrects the look-up table according to actual measurement, and stores the corrected values into the look-up table. The method is “non-invasive” in nature, but is a temperature prediction method rather than a real temperature measuring method. The results of the method are not results from real time measurements, but simply the estimated theoretical values under known conditions, therefore, it is unqualified to be used as evidence for temperature in clinic practice.

Accordingly, a non-invasive, effective real measuring method for the measurement of the temperature in treated region is highly desired in the field.

### SUMMARY OF THE INVENTION

The object of the invention is to provide a non-invasive, effective and practical method for the measurement of the temperature in the body of human (or animal), especially for the measurement of the high temperature (or low temperature) in the human (or animal) body generated by the High Intensive Focused Ultrasound (HIFU) that is utilized to react on the tissue with pathological changes in the treated region.

Of course, the method of this invention may also be utilized for the measurement of the high temperature (or low temperature) in the human (or animal) body generated with other means or methods (such as with radio frequency (RF) heating source or alternating current (AC) heating source).

Another object of the invention is to provide a non-invasive, effective apparatus for the practical clinical measurement applications to measure the temperature in the body of human (or animal), especially for the measurement of the high temperature (or low temperature) in the human (or animal) body generated by High Intensive Focused Ultrasound (HIFU) that is utilized to react on the tissue with pathological changes in the treated region.

The apparatus of this invention may also be utilized for the measurement of the high temperature (or low temperature) in the human (or animal) body generated with other means or methods (such as with radio frequency (RF) heating source or alternating current (AC) heating source).

The inventor creatively suggests an acoustic inversion measuring method to realize the above purposes.

In the method, a first ultrasonic wave is transmitted to a region to be measured, which has temperature T, under the guiding of M-mode ultrasound. The reflected ultrasonic wave from a particular reflection surface is received to obtain a first parameter. Then the temperature of the region to be measured is changed to T+ΔT. A second ultrasonic wave is transmitted to the region to be measured. The reflected ultrasonic wave from the second ultrasonic wave reflected by a particular reflection surface is received to obtain a second parameter. A measured comparison value between the second parameter and first parameter can be obtained. On the other hand, a theoretical comparison between the second parameter and first parameter can also be obtained through theoretical calculation. The differences between the theoretical comparison and measured comparison are processed by an optimization method. The local temperature increment ΔT of the region to be measured will be obtained with an inversion method. The invention also includes the corresponding apparatus to realize the above method and a focused ultrasonic therapeutic apparatus.

To explain the method of the invention, we will firstly discuss and establish the theory of the inventive method.

1. Echo Theory

The wave equation of ultrasound wave can be expressed as the following:

$${∇}^{2} p + {[ \frac{ω}{{C}_{0} + Δ C} ]}^{2} p = 0 , \qquad ( 1 )$$

wherein p is sound pressure; C₀ is the sound velocity at temperature T₀ (ambient temperature); ΔC is sound velocity increment when temperature increases ΔT; ω is angular frequency of the ultrasonic wave. As shown in the schematic drawing of FIG. 1. The O point at the center of sphere is the origin of coordinate, i.e., the center of the heated region, where the temperature increment has the largest value ΔTₘ. Therefore, one has

$$T = {T}_{0} + Δ {T}_{m} {ⅇ}^{- bR} \\ \frac{Δ C}{{C}_{0}} = αΔ {T}_{m} {ⅇ}^{- bR} , \\ α = \frac{1}{{C}_{0}} \frac{\partial C}{\partial T} \qquad ( 2 )$$

thus, formula (1) can be expressed approximately as:

$${∇}^{2} p + {k}^{2} p = 2 {k}^{2} \frac{Δ C}{{C}_{0}} p \\ k = ω / {C}_{0} \qquad ( 3 )$$

The pressure of scattered waves at a point B in the space can be obtained through the retarded solution of formula (3).

$${p}_{s} = - \frac{{k}^{2} αΔ {T}_{m}}{2 π} {\int }_{0}^{2 π} {\int }_{0}^{π} {\int }_{0}^{∞} {ⅇ}^{- bR} p \frac{{ⅇ}^{ⅈ kr}}{r} {R}^{2} sin ϑ ⅆ R ⅆ ϑ ⅆ φ \qquad ( 4 )$$

Assumed incident wave is
pᵢ=A₀e^{ikR cos,9}
Born Approximation will be used for the p under the sign of integration. Fresnel Approximation will be used for r. In this way, formula (4) can be approximately written as:

$$\begin{matrix}{p}_{s} = - {A}_{0} {k}^{2} αΔ {T}_{m} \frac{{ⅇ}^{ⅈ {kR}_{0}}}{{R}_{0}} {\int }_{0}^{∞} {R}^{2} {ⅇ}^{- bR + ⅈ \frac{{kR}^{2}}{2 {R}_{0}}} I ( R ) ⅆ R & ( 5 ) \\ I ( R ) = {\int }_{0}^{π} {ⅇ}^{ⅈ kR [ cos ϑ - cos ( ϑ - {ϑ}_{0} ) ] - ⅈ k \frac{{R}^{2}}{{R}_{0}} cos ( ϑ - {ϑ}_{0} )} sin ϑ ⅆ ϑ & ( 6 )\end{matrix}$$

where a, b is constant value; θ₀ is the included angle of <BOC, θ is the included angle of θAOC; OA=R, OB=R₀, r=AB. I(R) is proportional to the Fresnel integral. As for the factor e^{-bR} in formula (5), it is very small when bR is large, and mainly contributes the integral in the region of R≦1/b, thus kR≦k/b in formula (6). Because b is in the order of 10³ cm⁻¹ (electrical heating at 50 Hz) or in the order of 10⁴ cm⁻¹ (radio frequency heating), when the sound frequency f is in the order of 2 MHz, k will be in the order of 80 (cm⁻¹). Therefore, kR<<1, R/R₀<<1 in formula (6). With these approximations, the exponential term in formula (6) can be expanded to a series up to a first order approximation, the result is substituted into formula (5), the integral of formula (5) is obtained by means of the saddle point approximation and the final results are:

$${p}_{s} = 2 \sqrt{π} {A}_{0} α {b}^{2} {R}_{0}^{1.5} Δ {T}_{m} ( 1 + i ) [ 2 - \frac{π}{2} {bR}_{0} sin {ϑ}_{0} + i \frac{{b}^{2} {R}_{0}}{k} ( \frac{2}{3} - 2 {sin}^{2} {ϑ}_{0} ) ] exp \{ {ikR}_{0} [ 1 + \frac{{b}^{2}}{2 {k}^{2}} ] \} / \sqrt{k} \qquad ( 7 )$$

It corresponds to the scattered power:

$${W}_{s} = 2 π {A}_{0}^{2} {α}^{2} {b}^{4} {{R}_{0}^{3} ( Δ {T}_{m} )}^{2} [ {( 2 - \frac{π}{2} {bR}_{0} sin {ϑ}_{0} )}^{2} + {( \frac{{b}^{2} {R}_{0}}{k} )}^{2} {( \frac{2}{3} - 2 sin {ϑ}_{0} )}^{2} ] / k \qquad ( 8 )$$

The directional pattern of the scattered power is illustrated in FIG. 2.

It shows that the scattered power along the direction of θ₀=π/2 is far more larger than the scattered power along the direction of incident wave.

On the other hand, the formula (7) can be rewritten as:

$$\begin{matrix}{p}_{s} = \vert {p}_{s} \vert {ⅇ}^{ⅈ [ {kR}_{0} + Φ ]} \\ \vert {p}_{s} \vert = \sqrt{2 π} {A}_{0} α {b}^{2} {{R}_{0}^{1.5} [ {( 2 - \frac{π}{2} {bR}_{0} sin {ϑ}_{0} )}^{2} + {( \frac{{b}^{2} {R}_{0}}{k} )}^{2} {( \frac{2}{3} - 2 sin {ϑ}_{0} )}^{2} ]}^{\frac{1}{2}} & ( 9 ) \\ Φ = \frac{{b}^{2} {R}_{0}}{2 k} + arctan [ ( \frac{{b}^{2} {R}_{0}}{k} ) \frac{\frac{2}{3} - 2 {sin}^{2} {ϑ}_{0}}{2 - \frac{π}{2} {bR}_{0} sin {ϑ}_{0}} ] + \frac{π}{4} & ( 10 )\end{matrix}$$

All the signals are reflected by the reflection surface at D. The reflected signals are again going through the high temperature region and scattered one more time, they are received by the transducer after reaching point F (refer to FIG. 3). The final sound pressure of the echo can be obtained with formula (7), (9) and (10):
\overline{p}=\overline{p}₀S(β₁,R₀)S(β₂,L) (11)
wherein S(β, X), βⱼ (j=1,2) are complicated functions containing frequency f and ΔTₘ; \overline{p}₀=VA₀e^{ik(L+R}^{₀}⁾ is the sound pressure when there is no temperature gradient field; L and R₀ represent the distance from the transducer and reflection plane to the center of the hot source respectively, which can be measured each measurement by a B-scan ultrasonic apparatus. Formula (11) is the required expression for the sound pressure of the echo wave.

It is defined

$${I}_{1} ( {β}_{1} , {β}_{2} , … , Δ {T}_{m} , f ) = {( \frac{\overline{p}}{{\overline{p}}_{0}} )}^{2} \qquad ( 12 )$$

2. Measurement of Echo Wave and FFT Processing

The schematic diagram of the measuring method of the present invention is shown in FIG. 3. A transducer, which can be either a B-scan ultrasonic probe or a separate transducer, is installed at the plane going through point F, being used as both transmitter and receiver. If it is a separate transducer, it may be installed at the spherical shell of the ultrasonic source of HIFU apparatus, or at the head of the B-scan ultrasonic probe. The sphere between transducer and reflection plane is the heated region, the center of which has the highest temperature. It can be the focus of HIFU or the location to be heated by other heating source (such as RF or AC heating source). The plane going through D is the reflection plane. As known for those skilled in the art, this plane can always be found out, for instance, being determined by M-line ultrasound. Firstly, the transducer at point F sends out a sound wave before the region being heated. The sound wave is reflected to point F after it reaches the reflection plane. The transducer receives an echo wave, i.e., the sound pressure p₀ of the echo wave without going through a changed temperature field (referred to as First Echo Wave Parameter hereinafter). Then a heating source (not shown) generates heat and forms a temperature field. The ultrasonic wave going there through is scattered. The scattered wave superposes upon the transmitted wave, both of which are reflected after reaching the refection plane at point D. The reflected wave goes through the heated region again and is scattered another time before reaching point F. The transducer thus receives the sound pressure p₁ of the echo wave subjected to heating (referred to as Second Echo Wave Parameter hereinafter). These two echo waves carry the physical information of the heated region, especially the information on the temperature. The temperature information can be extracted after signal processing.

These two echo waves are subjected to FFT (Fast Fourier Transform) process. After smoothing the spectrum, the resulted sound pressure spectrums in the frequency domain is p₀(fᵢ) and p₁(fᵢ) respectively. The I₀(fᵢ) is defined as:

$${I}_{0} ( {f}_{i} ) = {[ \frac{{p}_{1} ( {f}_{i} )}{{p}_{0} ( {f}_{i} )} ]}^{2} \qquad ( 13 )$$

i=1, . . . , N, N is the number of the frequencies selected.

3. Optimum Processing and Inversion For Temperature.

An objective function is defined as:

$$Q = {\sum }_{i = 1}^{N} {\{ {I}_{0} ( {f}_{i} ) - {I}_{1} ( {β}_{1} , {β}_{2} , … , Δ {T}_{m} , {f}_{i} ) \}}^{2} \qquad ( 14 )$$

select β₁, β₂ . . . and ΔTₘ to make Q as the minimum value. Then the corresponding ΔTₘ is the difference between the temperature at the center of the location of the heated region and the ambient temperature T₀.

In above inversion derivation process, the values of the measured echo parameters are obtained at different frequencies through FFT processing. Then the minimum values of Q at all the frequencies are obtained with, for instance, the least square method. Then ΔTₘ is obtained through inversion. It should be understood by those skilled in the art that other mathematical processing methods may also be utilized to obtain proper ΔTₘ value as long as the optimizing processing for the differences between theoretical values and measured values is performed.

4. Empirical Formula

According to the results of formula (7)-(10) in above deduction, because part of the power is scattered due to the existence of high temperature region, the scattered wave has a phase shift in comparison with the incident wave (as indicated in formula (10)). In addition, the scattered wave also has a very complicated amplitude spectrum, which turns into 1.5 exponential from 0.5 exponential. Therefore, in practice, we may use empirical formulas derived to simplify the processing and calculation. In formula (11):
\overline{p}=\overline{p}₀S(β₁,R₀)S(β₂,L) (11)
S(β, X) is a complicated function of frequency f which is difficult to be calculated: It is firstly to smooth the measured spectrum of echo wave \overline{p}, then with further theoretical analysis, the measured results from acoustic measuring is made to conform to the measured results with other methods, in this way, following empirical formula is obtained, for instance

$$S ( β , X ) = 1 - \frac{β {X}^{3}}{f} \qquad ( 15 )$$

where
βⱼ=β₀ⱼΔTₘg(f,ΔTₘ), β₀ⱼ=(αb³C₀)ⱼ (16)
\overline{p}₀=VAₒe^{ik(L+R}^{₀}⁾ (17)
\overline{p}₀ is sound pressure of the echo wave when there is no temperature gradient field; g is a value to be determined; L and R₀ represent the distance from the transducer and reflection plane to the center of the hot source respectively, which can be measured by B-scan ultrasonic apparatus. Accordingly, formula (12) can be defined as:

$${I}_{1} ( {β}_{01} , {β}_{02} , … , Δ {T}_{m} , f ) = {( \frac{\overline{p}}{{\overline{p}}_{0}} )}^{2} \qquad ( {12}^{′} )$$

the objective function may be accordingly defined as:

$$Q = {\sum }_{i = 1}^{N} {\{ {I}_{0} ( {f}_{i} ) - {I}_{1} ( {β}_{01} , {β}_{02} , … , Δ {T}_{m} , {f}_{i} ) \}}^{2} \qquad ( {14}^{′} )$$

in which β₀₁, β₀₂ . . . are acoustic-thermal coupling parameters, which are dependent on temperature T and ΔTₘ. According to the experiments, they will be reduced with the increase of temperature and thus can be written generally as:

$${β}_{0 j} = {\sum }_{i = 0}^{M} {α}_{ij} ( T ) {( Δ {T}_{m} )}^{i} \qquad ( 18 )$$

The appropriate value of M and αᵢⱼ(T) can be obtained as the above way. Due to the lack of the measured data of αᵢⱼ, following method may be used to establish the relation between β₀ⱼ and αᵢⱼ. For instance, we select
β₀ⱼ=β₀ⱼ⁽⁰⁾(ΔTₘ)[1+Δ] (19)
β₀ⱼ=β₀ⱼ⁽⁰⁾(ΔTₘ)[1+Δ] indicates that it is dependent on ΔTₘ. Δ is a specified finely turned variance, for example, specifying −Δ₀≦Δ≦Δ₀, taking Δ₀=0.2. The β₀ⱼ⁽⁰⁾ and ΔTₘ are changing at certain interval in a relative wide range. During the data processing, firstly, a set of β₀ⱼ⁽⁰⁾ (j=1,2) may be specified. Then the ΔTₘ will be changed in a certain range at a certain interview, such as 5°, 10°, 15°, . . . Then formula (14′) may be utilized for calculation. During the calculation, Δ may be finely searched in the range of ±Δ₀. A minimum value of Q is given after the calculation. Secondly, another set of initial value for β₀ⱼ⁽⁰⁾ may be specified (with a certain changes at interval that is different from previous ones), and the ΔTₘ is changed (5°, 10°, 15°, . . . ) and making fine search for Δ. As such, another minimum Q value is obtained. In this way, make β₀ⱼ⁽⁰⁾ changing in a certain range and repeat above procedures, which will give a minimum value of Q every time. The least Q value will be selected from these minimum Q's. The β₀ⱼ and ΔTₘ corresponding thereto is the value required.

It should be understood that the above mathematical formulas and empirical formulas will not limit the present invention. Those skilled ones in the art may find other formulas with more advantages such as having quick calculation speed. The important idea of the present invention is the inversion approach for optimum difference between the theoretical values and the measured values, which will not be limited to the specified mathematical forms.

The inventor has made a large amount of measurements on the in vitro tissue and living body (such as pigs and rabbits), and compared the results with those from the other means and methods (such as RF or AC heating and measurement), The inventor also made comparisons and measurements during the clinical radio frequency treatments on liver cancer. The results have demonstrated the effectiveness and the accuracy of the invention (see the comparison tables below for detail).

The real-time measurement and control of the temperature in treated region during ultrasonic therapy is always a problem existing in the field. Some researchers even believe such a measurement is impossible to be realized. Such condition hinders the clinical popularization and application of this kind of therapy technology to some extent. The present invention is the first to suggest a method measuring the temperature of the focus in the body of human or animal with acoustic inversion approach. It is distinguished from the theoretical prediction method or look-up table method in that it is a real measurement. The invention utilizes the temperature information carried with ultrasonic echo wave, the temperature information in echo wave is extracted with optimum processing and inversion. The present invention thus resolves the pending problem of the real-time measurement of the temperature in treated region. This will certainly advance the development of the HIFU therapy and related technology.

To summarize the above, according to a first aspect of the invention, it provides a method for measuring the local temperature in the body of human or animal, characterized in that, comprising the following steps:
(1) after determination of the reflection plane with M-type ultrasound (M-line), transmitting a first ultrasonic wave to the region to be measured along the direction indicated by M-type ultrasound, receiving the echo wave of the first ultrasonic wave and obtaining a first echo wave parameter;
(2) changing the temperature of the region to be measured;
(3) transmitting a second ultrasonic wave to the region to be measured along the same direction, receiving the echo wave of the second ultrasonic wave and obtaining a second echo wave parameter, and calculating a comparison value between the second echo wave parameter and first echo wave parameter;
(4) according to theoretical calculation, obtaining a theoretical comparison value between the second echo wave parameter and first echo wave parameter;
(5) optimizing the differences between the theoretical comparison values and the measured comparison values, and obtaining the local temperature in the region to be measured by means of inversion.

According to a second aspect of the invention, an apparatus for the measurement of the local temperature changes in the body of human or animal is provided. The apparatus is characterized by including: an ultrasonic transmitting means that is used to transmit a first ultrasonic wave to the target region before the temperature of the target region is changed and transmit a second ultrasonic wave to the target region after the temperature of the target region is changed; an ultrasonic receiving means that is used to receive a first and a second echo wave from the first and second ultrasonic wave respectively reflected by the tissue of human or animal in the target region as well as the tissue away from the target region to obtain a first echo wave parameter and a second echo wave parameter respectively; a signal processing and analyzing means that is used to extract the temperature variation information of the target region from the first and second echo wave parameter. Wherein the signal processing and analyzing means calculates a theoretical comparison value between the first and second echo wave parameter, and optimizing the difference between the theoretical comparison value and the measured comparison value between the first and second echo wave parameters, then the information of the local temperature changes of the target region is obtained by means of inversion.

According to a third aspect of the invention, it provides an apparatus for the measurement of the local temperature changes in the body of human or animal. The apparatus including: an ultrasonic transmitting and receiving means, which is used to transmit a first ultrasonic wave with B-type ultrasound to the target region along the direction indicated by M-line before the temperature of the target region changes and receive a first echo wave from the first ultrasonic wave reflected by the tissue in the target region and the tissue away from the target region. After the temperature of the target region is changed, it transmit a second ultrasonic wave with B-type ultrasound to the target region along the direction indicated by M-line and receive a second echo wave from the second ultrasonic wave reflected by the tissue in the target region and the tissue away from the target region. In this way, it obtains a first and a second echo wave parameters respectively. The apparatus also includes a signal processing and analyzing means which extracts temperature change information of the target region from the first and second echo wave parameters. The signal processing and analyzing means calculates a theoretical comparison value between the first and second echo wave parameters with theoretical calculation and optimize the difference between the theoretical comparison value and the measured comparison value between the first and second echo wave parameters. Then the information of the local temperature changes of the target region can be obtained by means of inversion method.

According to a fourth aspect of the invention, it provides a focused ultrasonic therapeutic apparatus that can measure the temperature of the treated region. The apparatus includes a high-energy focusing ultrasonic source, which is used to generate high-energy focused ultrasound to a particular region of human body so as to change the temperature of the region; a positioning system which is used to move the region of human body to the focus of the high energy ultrasound and includes a B-type ultrasonic probe for positioning for the imaging of the particular region of the human body. The focused ultrasonic therapeutic apparatus includes following: at least one ultrasonic transducers for temperature measurement, which are installed at one side or both sides of the B-type ultrasonic probe for positioning, for transmitting a first ultrasonic wave to the particular region before the temperature of the region is changed and then receiving a first echo wave reflected by the tissue in the target region and the tissue away from the target region, and for transmitting a second ultrasonic wave to the particular region after the temperature of the region is changed and then receiving a second echo wave reflected by the tissue in the target region and the tissue away from the target region. In this way, a first and a second echo wave parameters are obtained. The apparatus also includes a signal processing and analyzing means which extracts temperature change information of the particular region from the first and second echo wave parameter. The signal processing and analyzing means calculates a theoretical comparison value between the first and second echo wave parameters with theoretical calculation and optimizes the difference between the theoretical comparison value and the measured comparison value between the first and second echo wave parameters, then the information of the local temperature changes of the target region can be obtained by means of inversion.

According to a fifth aspect of the invention, it provides another focused ultrasonic therapeutic apparatus that can measure the temperature. The apparatus includes a high-energy focusing ultrasonic source, which is used to generate high-energy focused ultrasound to a particular region of human body to change its temperature. It also includes a positioning system that is used to move the region of human body to the focus of the high energy ultrasound. The positioning system includes a B-type ultrasonic probe for positioning for the imaging of the particular region of the human body. This focused ultrasonic therapeutic apparatus is characterized in that, under the B/M mode, the positioning ultrasonic probe transmits a first ultrasonic wave to the particular region before the temperature of the region is changed along the direction indicated by M-line, and then receives a first echo wave reflected by the human tissue in the target region and the tissue away from the target region. The positioning ultrasonic probe is also used to transmit a second ultrasonic wave to the particular region after the temperature of the region is changed, and then receives a second echo wave reflected by the human tissue in the target region and the tissue away from the target region. In this way, a first and a second echo wave parameters are obtained. The apparatus also includes a signal processing and analyzing means which extracts temperature change information of the particular region from the first and second echo wave parameters. It calculates the ratio of the first and second echo wave parameters with theoretical calculation and optimizes objection function which involves a sum of square of the difference between the theoretical value and the measured value between the first and second echo wave parameters for all frequencies in the spectra, then the information of the local temperature changes of the target region can be obtained by means of the inversion method.

### DESCRIPTION OF THE ATTACHED DRAWINGS

FIG. 1 is a schematic diagram shows the wave theory of an embodiment according to the invention.

FIG. 2 is a directional pattern of the ultrasonic scattered power calculated in line with the theory.

FIG. 3 is a schematic diagram for the measurement of echo wave in an embodiment according to the invention.

FIG. 4A shows a schematic diagram for a practical measuring apparatus with HIFU heating source as one of the implementation examples of the invention.

FIG. 4B shows a schematic diagram for a practical measuring apparatus with HIFU heating source as another implementation example of the invention.

FIG. 5 shows a schematic diagram for the signal collection and processing in an embodiment according to the invention.

FIG. 6 shows the flow chart for the measurement procedures of an embodiment according to the invention.

FIG. 7 shows another implementation example of the temperature measurement probe of the invention. It shows the transmitter and receiver installed on the focused ultrasonic source of the therapeutic apparatus.

FIG. 8 shows another implementation example of the temperature measurement probe of the invention. It shows the transmitting and receiving means installed on both sides of the B-type ultrasonic probe for positioning the therapeutic apparatus.

FIG. 9 shows another implementation example for the temperature measurement probe of the invention. It shows the focused ultrasonic source of the therapeutic apparatus and the positioning B-type ultrasonic probe on the source. The B-type ultrasonic apparatus is modified so that the signal received therefrom may be used to subject to processing and analyzing for the temperature variation.

FIG. 10 illustrates the temperature verification and calibration with radio frequency heating source or AC heating source.

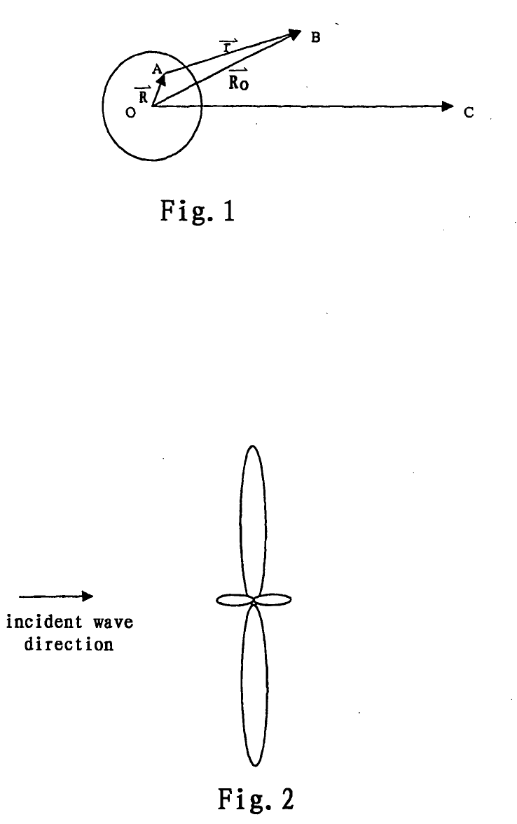

### PREFERRED EMBODIMENTS

The apparatus and measuring method of embodiments according to the invention will be described with reference to the attached drawings.

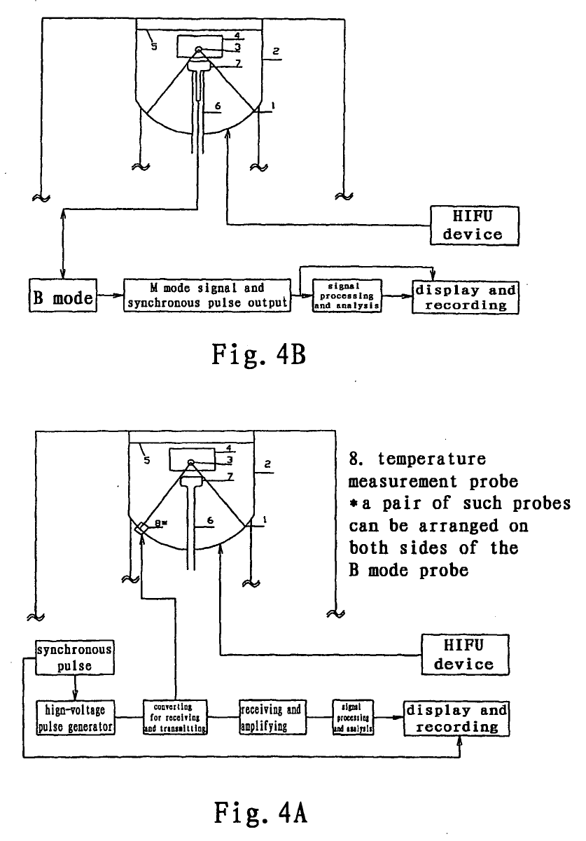

FIG. 4A and FIG. 4B are schematic diagrams showing exemplified HIFU heating and temperature measuring apparatus in accordance with an embodiment of the invention.

A practical in vitro focused ultrasonic therapeutic apparatus is typically comprised of following parts:
A. a high energy focusing ultrasonic source and its driving circuit for the generation of the high energy focused ultrasound.
B. a positioning system for locating the treated target on patient and moving it to the focus of the ultrasonic transducer, including a medical imaging system (mostly B-type ultrasonic apparatus), an means carrying the patient (such as a table or bed) and a moving system for the relative movement between the apparatus and the ultrasonic source.
C. a high-energy ultrasonic conductive structure and processing system of conductive medium: In the High Intensive Focused Ultrasound (HIFU) system, the ultrasound applied has to be transmitted into the patient body through special conductive medium (mostly using deaerated water). Therefore, a structure for accommodating the conductive medium (such as a water tank or water bag, etc.) as well as those for introducing and discharging the conductive medium are typically installed before the transmitting end of the high energy focused ultrasonic source.

The detailed descriptions for the known HIFU therapeutic apparatus will be omitted. The apparatus for the real-time monitoring of the temperature increment at the focus will be discussed in detail thereinafter.

The apparatus for the real-time monitoring of the temperature increment at the focus according to the invention mainly includes the follows:

1. Transmitting and Receiving Means for the Ultrasonic Pulse.

The apparatus may be one or one set of the ultrasonic transducers and the related transmitting and receiving circuit. The transducer(s) transmit ultrasonic pulses in the direction toward the focus of the high-energy focused ultrasonic therapeutic apparatus and receive the reflected ultrasonic wave being reflected from the tissue at the focus or the tissue away from the focus.

The medical B-type ultrasonic apparatus for positioning may act as the transmitting and receiving means in the guidance with the M-type ultrasound.

2. System for Processing & Analysis of the Reflection Wave Received.

After selecting appropriate part in the reflection wave signals, the system will make spectrum analysis. The results will be compared with the spectrum before the radiation of HIFU so as to obtain the information related to the changes of the temperature. The temperature variation (temperature difference) will be calculated and displayed.

Referring to FIG. 4A, the HIFU main body includes a water container 2, a temperature measurement sample 4 (human or animal) that is immersed under the water surface 5. The focused ultrasonic heating source 1 aims at the particular part of sample 4 (acoustic focus 3), and generates high-energy focused ultrasound for the heating or therapy and raises the temperature thereof. As part of the positioning system, positioning B-type ultrasonic probe 7 is under the control of the raising and lowering lever 6 to locate the sample target or move it to the focus of ultrasonic transducer. The HIFU system further includes a means for carrying the patient (such as a bed or table) and a moving system (not shown) for the relative movement between the means and the ultrasonic source.

In the HIFU system shown in FIG. 4A, an ultrasonic temperature probe 8 is included. It can be one or one group of ultrasonic transducers with related transmitting and receiving circuit. The transducer or transducers can transmit ultrasonic pulses in the direction toward the focus of the high-energy focused ultrasound and receive the reflection ultrasonic wave reflected from the tissue at the focus or from the tissue away from the focus. The ultrasonic temperature probe will be described in detail.

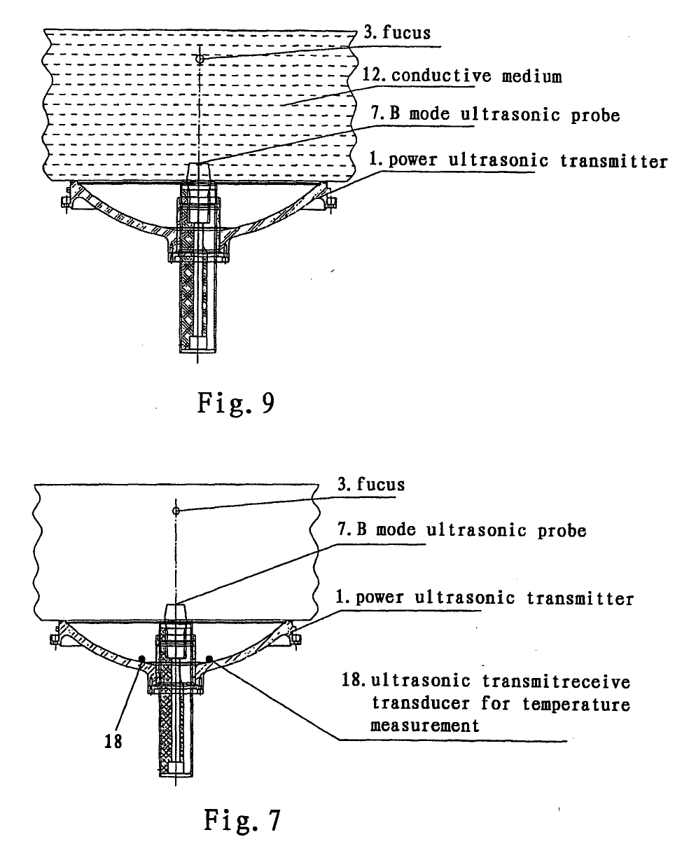

FIG. 7 shows a more detailed structure on which the ultrasonic temperature probe of the invention is installed. The ultrasonic temperature probe 8 includes two ultrasonic transducers 18. One of them is used for transmitting ultrasonic pulses in the direction toward the focus 3, another is used to receive the reflection wave reflected by the tissue in and away from the focus. Both are installed on the casing shell of the HIFU apparatus and each on one side of the positioning B-type ultrasound probe 7 respectively. In this way, the ultrasound paths for the ultrasonic temperature probe 8, the positioning ultrasonic probe and the ultrasound source for heating are separated with each other. It is also possible to use just one ultrasonic transducer 18 installed at one side of the positioning B-type ultrasonic probe 7 and used both for transmitting the ultrasonic pulses and receiving the reflection wave reflected from the tissue in and away from the focus 3.

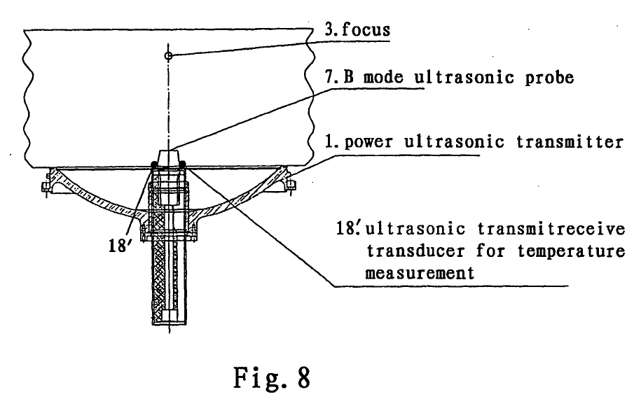

FIG. 8 shows another kind of structure for the ultrasonic temperature probe 8 to be installed on the system. The ultrasonic temperature probe 8 shown in this figure includes two ultrasonic transducers 18′. They are directly but separately installed on the head of the positioning B-type ultrasonic probe. In this way, the ultrasonic signal for temperature measurement may be directly located onto the focus by means of the movement of the positioning B-type ultrasonic probe. Similarly, it is also possible to use just one ultrasonic transducer 18′ installed at one side of the positioning B-type ultrasonic probe and used both for transmitting the ultrasonic pulses and receiving the reflection wave reflected from the tissue in and away from the focus 3.

FIG. 9 shows another kind of structures for the ultrasonic temperature probe 8 to be installed on the system. The ultrasonic probe 7 for positioning in the apparatus is directly used under B/M mode as the probe for transmitting the ultrasonic pulses and receiving the reflected ultrasonic signals. That is, the B-type ultrasonic probe 7 transmits ultrasonic wave in the direction guided by the M-type ultrasound, and the reflected wave signal received by the B-type ultrasonic probe is directly utilized to make analysis. This structure further simplifies the design and reduces the manufacturing cost for the apparatus. This implementation example demonstrates an additional advantage of the invention when the B-type ultrasonic probe is used to act as ultrasonic temperature probe in B/M mode: there is no additional hardware is needed, the existing HIFU apparatus can be used to realize the inventive temperature measuring method.

Return to FIG. 4A, the ultrasonic temperature probe 8 is coupled to a high-voltage pulse generator and a transmitting and receiving switching circuit which are under the control of a synchronization pulse circuit, transmitting and receiving of the temperature measurement pulse. The received echo wave signal is processed in a receiving and amplifying circuit. The measured value will be transmitted to the signal processing and analyzing system (for example, a computer connected to the apparatus) of the invention for processing and analyzing. The final results are shown on a displaying and recording means (such as the monitor of the computer). The signal processing and analyzing system may include the software for the calculation of the temperature inversion method of the invention. This system will be further explained in the following portions.

When the ultrasonic temperature probe adopts the arrangement in FIG. 9, the system design may be modified as another structure shown in FIG. 4B. The positioning function and temperature measurement function may share one signal extracting circuit for both B-type and M-type ultrasound. The positioning function will directly display the B-type ultrasonic signal on the displaying and recording means (such as the monitor of the computer). And the temperature measuring function will send the received signals to the signal processing and analyzing system (for example, the computer) for processing and analyzing. The final results are shown on the displaying and recording means (such as the monitor of the computer).

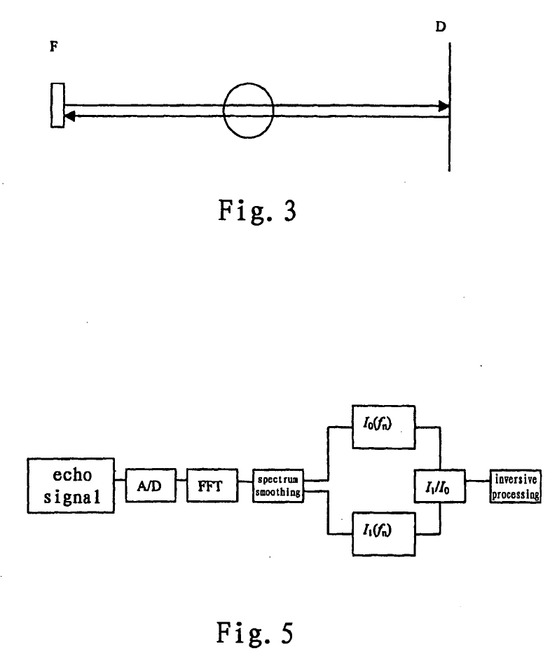

The measurement procedures of the invention are summarized in FIG. 5. First, the temperature is not raised due to the focused ultrasonic heating source 1 is still off. The ultrasonic temperature probe 8 (or the positioning B-type ultrasound probe 7 which is directly used as the ultrasonic temperature probe as described above) will transmit ultrasonic wave. The ultrasonic wave is reflected by the tissue on the focus or the tissue away from the tissue and the ultrasonic temperature probe 8 will receive a echo wave, i.e., the echo wave I₀ without subjecting to the temperature gradient field (corresponding to the first echo wave parameter). The reflection plane D of the echo wave (in FIG. 3) can be determined by the M-type ultrasound processing circuit. For example, a reflection plane is typically shown on the screen of the monitor. The operator (e.g. clinical doctor) thus can get the value of L and R₀ measured. When the B-type ultrasonic apparatus is operated under the B/M mode, a M-line is shown on the screen typically as a dotted line, rotating the M-line to make it go through the focus and intersected with the reflection plane. In this way, it ensures that the transmitted signal and reflected signal will both go through the heated region centered on the focus. Such a specific operation is well known in the art and will not be described in detail.

Then the focused ultrasonic heating source 1 is turned on to heat and build a temperature gradient field. The ultrasonic temperature probe 8 (or the positioning B-type ultrasound probe 7 which is directly used as the ultrasonic temperature probe as described above) will transmit ultrasonic wave again. The ultrasonic wave will be scattered when going through the temperature field. The scattered ultrasonic wave is superposed onto the transmitted wave. They are reflected upon reaching the reflection plane D. Then the reflected wave is scattered once again when going back through the heated region. Finally, the ultrasonic temperature probe 8 will receive an echo wave I₁ which has been subjected to heating (corresponds to the second echo wave parameter).

These two echo waves (I₀ and I₁) received by the ultrasonic temperature probe carry the physical information of the heated region, especially the temperature information. The information can be extracted after signal processing. This processing may be carried out by the signal processing and analyzing system.

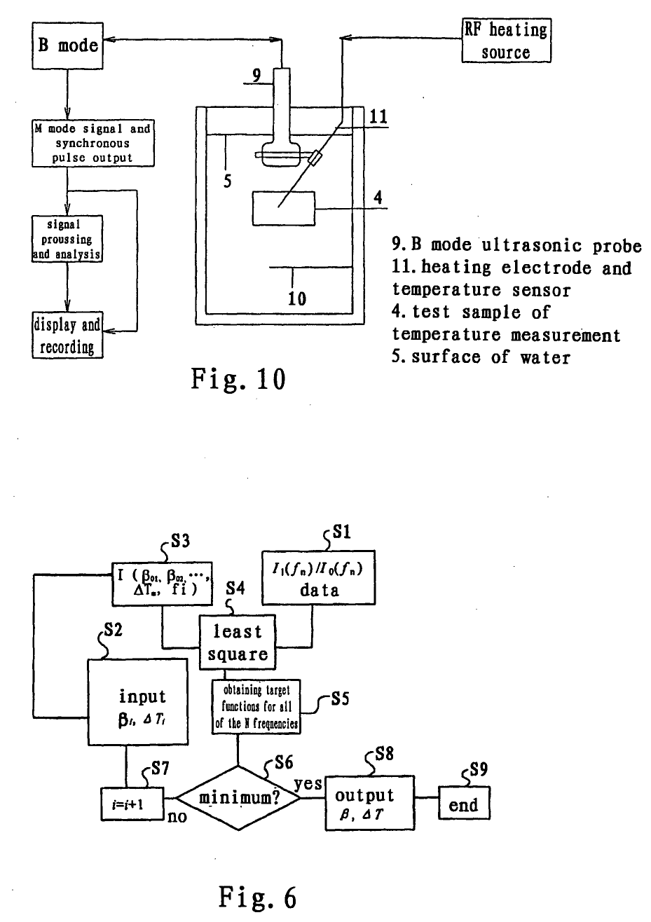

After A/D conversion, these two echo wave signals will be processed with FFT. Optionally, the result spectrum will be smoothed. Thus, the acoustic frequency spectrum will be obtained at the selected frequencies, i.e., the acoustic wave I₀(fᵢ) (i=1, . . . , N, N is the number of the frequencies selected) that does not go through the heated region, and the acoustic wave I₁(fᵢ) (i=1, . . . , N, N is the number of the frequencies selected) that does go through the heated region. The resulted values will be processed with inversion method based on the previously mentioned formula (7)-(14). As an example, the inversion processing flow chart based on the previously mentioned empirical formula is summarized in FIG. 6.

During the inversion processing, the operator (e.g. clinical doctor) will make a rough estimation on the temperature increment ΔTₘ at focus according to his/her experience and common knowledge in the art. For example, in the range of 10-50 C.°, the interval may be set as 1 C.°. When performing fine research, the interval may equal to or less than 0.1 C.°; β₀ⱼ=0.50, 0.45, 0.40, . . . etc. (refers to the descriptions following the formula (18) and (19)). First, the input means for the signal processing and analyzing system (for example, the keyboard of the computer connected to the apparatus, not shown in the figure) is used to input the initial ΔTₘ and β₀ⱼ value (step S2 in FIG. 6). The corresponding I₁(β₀₁,β₀₂, . . . ,ΔTₘ,fᵢ) is found according to formula (12) (step S3). The processing and analyzing system will find the I₁(fᵢ)/I₀(fᵢ) after data processing with the two echo wave signals, i.e., the

$$\frac{{p}_{1}^{2} ( {f}_{i} )}{{p}_{0}^{2} ( {f}_{i} )}$$

in formula (13) (step S1) which is then substituted into the objective function in formula (14) for inversion calculation (step S4, S5). Repeat the above input and processing steps (steps S6→S7→S2→S3, S1→S4→S5) until the minimum value of the objective function is obtained. The corresponding ΔTₘ to the minimum value of the objective function is the temperature increment at the focus. The temperature value is then output for display (S8), and the processing procedure is finished (S9).

It should be noted that the above data input procedure can also be automatically accomplished with the computer of the signal processing and analyzing. For example, the computer may automatically generate a plurality of data sets of ΔTₘ and β₀ⱼ values (e.g. followed the above described rules), and then, based on the measured I₀ and I₁ value, obtain the objective function at all the frequencies to find out the minimum value and obtain ΔTₘ with the inversion method.

FIG. 10 illustrates the temperature verification and calibration with radio frequency heating source or AC heating source. The reference sign 11 refers to the heating electrode and temperature sensor of the radio frequency heating source that is used to measure the focus temperature of test sample 4 (invasive measurement). The ultrasonic temperature probe 9 according to the present invention is also used (and can be used as a positioning probe simultaneously) to measure focus temperature of the test sample 4 at the same time. The description to the similar elements as those in FIG. 4 will be omitted. The illustrated arrangement can measure same temperature field with both the temperature sensor 11 (thermocouples) and the acoustic inversion method according to the invention. The parameters in the empirical formula can be calibrated by best matching the results from the two methods. It can also be used in verification or comparison between the two methods.

Table 1 and Table 2 respectively show the data comparison on the temperature of the living pig and human liver cancer tissue measured with the acoustic inversion method and radio frequency measuring method (thermocouples).

TABLE 1
A comparison of results between temperatures measured
with the acoustic inversion method and radio frequency
measuring method(thermocouples)

| Sample | No. | T₀(° C.) | ΔT° C. | T(AI)° C. | T(RF)° C. |
|---|---|---|---|---|---|
| Kidney | 14/15 | 39 | 12.8 | 51.8 | 53 |
| | 12/13 | 40 | 31.2 | 71.2 | 76 |
| | 16/17 | 40 | 37.2 | 77.2 | 77 |
| | 18/19 | 39 | 45 | 84 | 87 |
| | 20/21 | 40 | 58.5 | 98.5 | 104 |
| Liver | 22/23 | 39 | 14.1 | 53.1 | 53 |
| | 24/25 | 40 | 25.9 | 65.9 | 65 |
| | 26/27 | 40 | 42.4 | 82.4 | 83 |
| | 28/29 | 41 | 46.7 | 87.7 | 93-87 |
| | 30/31 | 41 | 56 | 97 | 96 |

T₀: body temperature of pig
ΔT: the increase of temperature after heating
T(AI): temperature measured with the acoustic inversion method
T(RF): temperature measured with radio frequency measuring method(thermocouples)

TABLE 2
A comparison of results between temperatures of a liver
cancer patient measured with non-invasive measuring method
and radio frequency measuring method(thermocouples)

| No. | T₀(° C.) | ΔT° C. | T(AI)° C. | T(RF)° C. |
|---|---|---|---|---|
| 0/1 | 37.5 | 12.2 | 47.2 | 45-52 |
| 0/2 | 37.5 | 31.4 | 68.9 | 64-74 |
| 0/3 | 37.5 | 48.9 | 86.4 | 79-97 |
| 4/5 | 42-43 | 11.1 | 53.1-54.1 | 48-54 |
| 4/6 | 42-43 | 21.5 | 63.5-64.5 | 62-68 |
| 4/7 | 42-43 | 49.3 | 91.3-92.3 | 71-111 |
| 8/9 | 55 | 14 | 69 | 70-73 |
| 8/10 | 55 | 27.8 | 82.8 | 79-82 |
| 8/11 | 55 | 36 | 91 | 90-95 |
| 12/13 | 60-66 | 19.4 | 79.4-85.4 | 71-85 |
| 12/14 | 60-66 | 22.2 | 82.2-88.2 | 79-98 |
| 12/15 | 60-66 | 27.5 | 87.5-93.5 | 85-107 |
| 16/17 | 60 | 13.5 | 73.5 | 70-72 |
| 16/18 | 60 | 27.5 | 87.5 | 81-83 |
| 16/19 | 60 | 35 | 95 | 88-94 |
| 20/21 | 53.5 | 28.2 | 81.7 | 68-75 |
| 20/22 | 53.5 | 28 | 81.5 | 77-85 |
| 20/23 | 53.5 | 40.5 | 94 | 86-97 |

T₀: body temperature of the patient
ΔT: the increase of temperature after heating
T(AI): temperature measured with the acoustic inversion method
T(RF): temperature measured with radio frequency measuring method(thermocouples)

The present invention has been described with specific embodiments with reference to the attached drawings. However, it should be understood that this invention is not limited to the specific modes of the above embodiments. For example, the structure of the apparatus itself may have various modifications; from the principle point of view, this invention not only can measure the local temperature increase relevant to ambient temperature, but also can measure the local temperature decrease.

1. A method for measuring the local temperature in the body of human or animal, characterized in that, comprising the following steps:
(1) transmitting a first ultrasonic wave to the region to be measured, receiving the echo wave of the first ultrasonic wave and obtaining a first echo wave parameter;
(2) changing the temperature of the region to be measured;
(3) transmitting a second ultrasonic wave to the region to be measured, receiving the echo wave of the second ultrasonic wave and obtaining a second echo wave parameter, and calculating a measured comparison value between the second echo wave parameter and the first echo wave parameter;
(4) through theoretical calculation, obtaining a theoretical comparison value between the second echo wave parameter and the first echo wave parameter;
(5) optimizing the differences between the theoretical comparison values and the measured comparison values, thereby obtaining the local temperature in the region by means of inversion.

2. A method according to claim 1, wherein said first echo parameter and said second echo parameter are sound pressure of the echo wave or power of the echo wave.

3. A method according to claim 1, further comprises: determining the reflective plane of the ultrasound using M-mode ultrasound, performing said transmitting in a direction specified by the M-line ultrasound.

4. A method according to claim 1, wherein the formula used for calculating the theoretical comparison value between the second echo wave parameter and the first echo wave parameter is
\overline{p}=\overline{p}₀S(β₁,R₀)S(β₂,L) (11)
wherein the following empirical formulas are used:
$$S ( β , X ) = 1 - \frac{β {X}^{3}}{f} \qquad ( 15 )$$
βⱼ=β₀ⱼΔTₘg(f,ΔTₘ), (16)
\overline{p}₀=VA₀e^{ik(L+R}^{₀}⁾ (17)
wherein \overline{p}₀ is sound pressure of the echo wave when there is no temperature gradient field; \overline{p} is sound pressure of the echo wave when there is a temperature gradient field; f is the frequency of the sound wave; g is a value to be determined; L and R₀ represent the distance from the transducer and the reflection plane to the center of the hot source respectively; ΔTₘ is the maximum temperature increase at the center of hot source relevant to ambient temperature, and the comparison value between the first echo wave parameter and the second echo wave parameter is defined as
$${I}_{1} ( {β}_{01} , {β}_{02} , … , Δ {T}_{m} , f ) = {( \frac{\overline{p}}{{\overline{p}}_{0}} )}^{2} \qquad ( {12}^{′} )$$
in which β₀₁, β₀₂ . . . are acoustic-thermal coupling parameters.

5. A method according to claim 4, wherein the acoustic-thermal coupling parameter is expressed as
$${β}_{0 j} = {\sum }_{i = 0}^{M} {α}_{ij} ( T ) {( Δ T )}^{i} \qquad ( 18 )$$

6. A method according to claim 5, wherein the acoustic-thermal coupling parameter is further expressed as
β₀ⱼ=β₀ⱼ⁽⁰⁾(ΔTₘ)[1+Δ] (19)
in which Δ is a specified fine variance.

7. A method according to claim 1, wherein the optimum step includes performing fast Fourier transformation (FFT) and then spectrum smoothing to the measured first echo wave parameters and second echo wave parameters, and obtaining the minimum difference between the theoretical comparison values and the measured comparison values by means of the least square method so as to obtain the local temperature of the region to be measured by means of inversion.

8. A method according to claim 7, wherein the optimizing step can be expressed with the following formulas:
the sound pressure spectrum of the first echo wave parameter and the second echo wave parameter in the frequency domain are respectively p₀(fᵢ) and lᵢ(fᵢ), I₀(fᵢ) is defined as
$${I}_{0} ( {f}_{i} ) = {[ \frac{{p}_{1} ( {f}_{i} )}{{p}_{0} ( {f}_{i} )} ]}^{2} \qquad ( 13 )$$
i=1, . . . , N, N is the number the frequencies selected,
an objective function is defined as:
$$Q = {\sum }_{i = 1}^{N} {\{ {I}_{0} ( {f}_{i} ) - {I}_{1} ( {β}_{01} , {β}_{02} , … , Δ {T}_{m} , {f}_{i} ) \}}^{2} \qquad ( {14}^{′} )$$
selecting β₁, β₂ . . . and ΔTₘ to make Q as the minimum value, the corresponding ΔTₘ is the differential value between the temperature of the hot source and the ambient temperature T₀.

9. An apparatus for measuring the local temperature changes in the body of human or animal, characterized in that, comprising: an ultrasonic transmitting means used for transmitting a first ultrasonic wave to the target region before its temperature is change and transmitting a second ultrasonic wave to the target region after its temperature is changed; an ultrasonic receiving means used for receiving a first and a second echo waves from said first and second ultrasonic wave respectively reflected by the tissue of human or animal in the target region and the tissue away from the target region to obtain a first echo wave parameter and a second echo wave parameter respectively;
a signal processing and analyzing means used for extracting the temperature variation information of the target region from the first and second echo wave parameter;
wherein the signal processing and analyzing means calculates a theoretical comparison value between the first and second echo wave parameter, and optimizes the differences between the theoretical comparison values and the measured comparison values between the first and second echo wave parameters, such that the information of the local temperature changes of the target region can be obtained through inversion.

10. An apparatus according to claim 9, wherein said first echo parameter and said second echo parameter are sound pressure of the echo wave or power of the echo wave.

11. An apparatus according to claim 9, wherein the formula used for the signal processing and analyzing means to calculate the theoretical comparison value between the second echo wave parameter and the first echo wave parameter is
\overline{p}=\overline{p}₀S(β₁,R₀)S(β₂,L) (11)
wherein the following empirical formulas are used:
$$S ( β , X ) = 1 - \frac{β {X}^{3}}{f} \qquad ( 15 )$$
βⱼ=β₀ⱼΔTₘg(f,ΔTₘ), (16)
\overline{p}₀=VA₀e^{ik(L+R}0) (17)
\overline{p}₀ is sound pressure of the echo wave when there is no temperature gradient field; \overline{p} is sound pressure of the echo wave when there is a temperature gradient field; f is the frequency of the sound wave; g is a value to be determined, L and R₀ represent the distance from the transducer and the reflection plane to the center of the hot source respectively; ΔTₘ is the maximum temperature increase at the center of hot source relevant to ambient temperature, and the comparison value between the first echo wave parameter and the second echo wave parameter is defined as
$${I}_{1} ( {β}_{01} , {β}_{02} , … , Δ {T}_{m} , f ) = {( \frac{\overline{p}}{{\overline{p}}_{0}} )}^{2} \qquad ( {12}^{′} )$$
in which β₀₁, β₀₂ . . . are acoustic-thermal coupling parameters.

12. An apparatus according to claim 11, wherein the acoustic-thermal coupling parameter is expressed as
$${β}_{0 j} = {\sum }_{i = 0}^{M} {α}_{ij} ( T ) {( Δ T )}^{i} \qquad ( 18 )$$

13. An apparatus according to claim 12, wherein the acoustic-thermal coupling parameter is further expressed as
β₀ⱼ=β₀ⱼ⁽⁰⁾(ΔTₘ)[1+Δ] (19)
in which Δis a specified fine variance.

14. An apparatus according to claim 9, wherein signal processing and analyzing means performs fast Fourier transformation (FFT) and spectrum smoothing to the measured first echo wave parameter and second echo wave parameter, and obtains the minimum difference between the theoretical comparison values and the measured comparison values by means of the least square method so as to obtain the local temperature of the target region by means of inversion.

15. An apparatus according to claim 14, wherein obtaining the local temperature changes of the target region by the signal processing and analyzing means can be expressed with the following formulas:
the sound pressure spectrums of the first echo wave parameters and the second echo wave parameters in the frequency domain are respectively p₀(fᵢ) and p₁(fᵢ), I₀(fᵢ) is defined as
$${I}_{0} ( {f}_{i} ) = {[ \frac{{p}_{1} ( {f}_{i} )}{{p}_{0} ( {f}_{i} )} ]}^{2} \qquad ( 13 )$$
i=1, . . . , N, N is the number the frequencies selected,
an objective function is defined as
$$Q = {\sum }_{i = 1}^{N} {\{ {I}_{0} ( {f}_{i} ) - {I}_{1} ( {β}_{01} , {β}_{02} , … , Δ {T}_{m} , {f}_{i} ) \}}^{2} \qquad ( {14}^{′} )$$
selecting, β₁, β₂ . . . and ΔTₘ to make Q as the minimum value, the corresponding ΔTₘ is the differential value between the temperature of the heated location and the ambient temperature T₀.

16. An apparatus according to claim 15, wherein the signal processing and analyzing means comprises an input means used for inputting a plurality of data sets of β₀₁, β₀₂ . . . and ΔTₘ by the user.

17. An apparatus according to claim 15, wherein the signal processing and analyzing means automatically generates a plurality of data sets of β₀₁, β₀₂ . . . and ΔTₘ.

18. An apparatus for measuring the local temperature changes in the body of human or animal, characterized in that, comprising:
an ultrasonic transmitting and receiving means used for transmitting a first ultrasonic wave to a target region before its temperature is changed and receiving a first echo wave from said first ultrasonic wave reflected by the tissue of human or animal in the target region and the tissue away from the target region; and for transmitting a second ultrasonic wave to the target region after the temperature of the target region is changed and receiving a second echo wave from said second ultrasonic wave reflected by the tissue of human or animal in the target region and the tissue away from the target region thereby a first echo wave parameter and a second echo wave parameter are obtained respectively;
a signal processing and analyzing means used for extracting temperature change information of the target region from said first and second echo wave parameters,
wherein the signal processing and analyzing means calculates a theoretical comparison value between the first and second echo wave parameter, and optimizes the differences between the theoretical comparison values and the measured comparison values between the first and second echo wave parameters, such that the information of the local temperature changes of the target region can be obtained through inversion.

19. An apparatus according to claim 18, wherein said first echo parameter and said second echo parameter are sound pressure of the echo wave or power of the echo wave.

20. An apparatus according to claim 18, wherein the transmitting and receiving means conducts said transmitting by B-type ultrasound along the direction indicated by M-line.

21. An apparatus according to claim 18, wherein the formula used by the transmitting and receiving means for calculating the theoretical comparison value between the second echo wave parameter and the first echo wave parameter is
\overline{p}=\overline{p}₀S(β₁,R₀)S(β₂,L) (11)
wherein the following empirical formulas are used:
$$S ( β , X ) = 1 - \frac{β {X}^{3}}{f} \qquad ( 15 )$$
βⱼ=β₀ⱼΔTₘg(f,ΔTₘ), (16)
\overline{p}₀=VA₀e^{ik(L+R}^{₀}⁾ (17)
\overline{p}₀ is sound pressure of the echo wave when there is no temperature gradient field; \overline{p} is sound pressure of the echo wave when there is a temperature gradient field; f is the frequency of the sound wave; g is an value to be determined; L and R₀ represent the distance from the transducer and the reflection plane to the center of the hot source respectively; ΔTₘ is the maximum temperature increase at the center of hot source relevant to ambient temperature, and the comparison value between the first echo wave parameter and the second echo wave parameter is defined as
$${I}_{1} ( {β}_{01} , {β}_{02} , … , Δ {T}_{m} , f ) = {( \frac{\overline{p}}{{\overline{p}}_{0}} )}^{2} \qquad ( {12}^{′} )$$
in which β₀₁, β₀₂ . . . are acoustic-thermal coupling parameters.

22. An apparatus according to claim 21, wherein the acoustic-thermal coupling parameter is expressed as
$${β}_{0 j} = {\sum }_{i = 0}^{M} {α}_{ij} ( T ) {( Δ T )}^{i} \qquad ( 18 )$$

23. An apparatus according to claim 22, wherein the acoustic-thermal coupling parameter is further expressed as
β₀ⱼ=β₀ⱼ⁽⁰⁾(ΔTₘ)[1+Δ] (19)
in which Δis a specified fine variance.

24. An apparatus according to claim 18, wherein signal processing and analyzing means performs fast Fourier transformation (FFT) and spectrum smoothing to the measured first echo wave parameter and second echo wave parameter, and obtains the minimum difference between the theoretical comparison values and the measured comparison values by means of the least square method so as to obtain local temperature increment of the target region by means of inversion.

25. An apparatus according to claim 24, wherein obtaining the local temperature increment of the target region by the signal processing and analyzing means can be expressed with the following formulas:
the sound pressure spectrums of the first echo wave parameter and the second echo wave parameter in the frequency domain are respectively p₀(fᵢ) and p₁(fᵢ), I₀(fᵢ) is defined as
$${I}_{0} ( {f}_{i} ) = {[ \frac{{p}_{1} ( {f}_{i} )}{{p}_{0} ( {f}_{i} )} ]}^{2} \qquad ( 13 )$$
i=1, . . . , N, N is the number the frequencies selected,
An objective function is defined as
$$Q = {\sum }_{i = 1}^{N} {\{ {I}_{0} ( {f}_{i} ) - {I}_{1} ( {β}_{01} , {β}_{02} , … , Δ {T}_{m} , {f}_{i} ) \}}^{2} \qquad ( 14 )$$
selecting β₁, β₂ . . . and ΔTₘ to make Q as the minimum value, the corresponding ΔTₘ is the differential value between the temperature of the hot source and the ambient temperature T₀.

26. An apparatus according to claim 25, wherein the signal processing and analyzing means comprises an input means used for inputting a plurality of data sets of β₀₁, β₀₂ . . . and ΔTₘ by the user.

27. An apparatus according to claim 25, wherein the signal processing and analyzing means automatically generates a plurality of data sets of β₀₁, β₀₂ . . . and ΔTₘ.

28. A focused ultrasonic therapeutic apparatus that can measure the temperature, comprising:
a high-energy focused ultrasonic source used for generating high-energy focused ultrasound to a particular region of human body to change the temperature thereof;
a positioning system used for moving said particular region of human body to the focus of the high energy ultrasound, including a positioning B-type ultrasonic probe for imaging said particular region of human body;
characterized in that, the focused ultrasonic therapeutic apparatus further comprises:
at least one measuring ultrasonic transducers, which are installed at one side or both sides of the positioning B-type ultrasonic probe, used for transmitting a first ultrasonic wave to the particular region before the temperature of the region is changed, then receiving a first echo wave from said first ultrasonic wave reflected by the human tissue in the target region and the tissue away from the target region; and for transmitting a second ultrasonic wave to the particular region after the temperature of the region is changed, then receiving a second echo wave from said second ultrasonic wave reflected by the human tissue in the target region and the tissue away from the target region thereby a first echo wave parameter and a second echo wave parameter are obtained respectively;
a signal processing and analyzing means for extracting temperature changing information of the particular region from the first and second echo wave parameter;
wherein the signal processing and analyzing means calculates a theoretical comparison value between the first and second echo wave parameter, and optimizes the differences between the theoretical comparison values and the measured comparison values between the first and second echo wave parameters, such that the information of the local temperature changes of the particular region can be obtained through inversion.

29. A focused ultrasonic therapeutic apparatus according to claim 28, wherein said first echo parameter and said second echo parameter are sound pressure of the echo wave or power of the echo wave.

30. A focused ultrasonic therapeutic apparatus according to claim 28, wherein said at least one measuring ultrasonic transducers are located on a casing shell of the ultrasonic therapeutic apparatus which contains the conductive medium.

31. A focused ultrasonic therapeutic apparatus according to claim 28, wherein said at least one measuring ultrasonic transducers are located on the head of the positioning B-type ultrasonic probe, so that said at least one measuring ultrasonic transducers move together with the positioning B-type ultrasonic probe.

32. A focused ultrasonic therapeutic apparatus according to claim 28, wherein the formula used by the transmitting and receiving means for calculating the theoretical comparison value between the second echo wave parameter and the first echo wave parameter is
\overline{p}=\overline{p}₀S(β₁,R₀)S(β₂,L) (11)
wherein the following empirical formulas are used:
$$S ( β , X ) = 1 - \frac{β {X}^{3}}{f} \qquad ( 15 )$$
βⱼ=β₀ⱼΔTₘg(f,ΔTₘ), (16)
\overline{p}₀=VA₀e^{ik(L+R}^{₀}⁾ (17)
\overline{p}₀ is sound pressure of the echo wave when there is no temperature field; \overline{p} is sound pressure of the echo wave when there is a temperature field; f is the frequency of the sound wave; g is a value to be determined; L and R₀ represent the distance from the transducer and reflection plane to the center of the hot source respectively; ΔTₘ is the maximum temperature increase at the center of hot source relevant to ambient temperature, and the comparison value between the first echo wave parameter and the second echo wave parameter is defined as
$${I}_{1} ( {β}_{01} , {β}_{02} , … , Δ {T}_{m} , f ) = {( \frac{\overline{p}}{{\overline{p}}_{0}} )}^{2} \qquad ( {12}^{′} )$$
in which β₀₁, β₀₂ . . . are acoustic-thermal coupling parameters.

33. A focused ultrasonic therapeutic apparatus according to claim 32, wherein the acoustic-thermal coupling parameter is expressed as
$${β}_{0 j} = {\sum }_{i = 0}^{M} {α}_{ij} ( T ) {( Δ T )}^{i} \qquad ( 18 )$$

34. A focused ultrasonic therapeutic apparatus according to claim 33, wherein the acoustic-thermal coupling parameter is further expressed as
β₀ⱼ=β₀ⱼ⁽⁰⁾(ΔTₘ)[1+Δ] (19)
in which Δ is a specified fine variance.

35. A focused ultrasonic therapeutic apparatus according to any one of claim 28, wherein said signal processing and analyzing means performs fast Fourier transformation (FFT) and spectrum smoothing to the measured first echo wave parameter and second echo wave parameter, and obtains the minimum difference between the theoretical comparison values and the measured comparison values by means of the least square method so as to obtain the local temperature increment of the particular region by means of inversion.

36. A focused ultrasonic therapeutic apparatus according to claim 35, wherein the transmitting and receiving means obtains the temperature increment in the particular region by means of the inversion method, which can be expressed with the following formulas:
the sound pressure spectrums of the first echo wave parameter and second echo wave parameter in the frequency domain are respectively p₀(fᵢ) and p₁(fᵢ), I₀(fᵢ) is defined as
$${I}_{0} ( {f}_{i} ) = {[ \frac{{p}_{1} ( {f}_{i} )}{{p}_{0} ( {f}_{i} )} ]}^{2} \qquad ( 13 )$$
i=1, . . . , N, N is the number the frequencies selected,
An objective function is defined as
$$Q = {\sum }_{i = 1}^{N} {\{ {I}_{0} ( {f}_{i} ) - {I}_{1} ( {β}_{01} , {β}_{02} , … , Δ {T}_{m} , {f}_{i} ) \}}^{2} \qquad ( 14 )$$
selecting β₁, β₂ . . . and ΔTₘ to make Q as the minimum value, the corresponding ΔTₘ is the differential value between the temperature of the hot source and the ambient temperature T₀.

37. A focused ultrasonic therapeutic apparatus according to claim 36, wherein the signal processing and analyzing means comprises an input means used for inputting a plurality of data sets of β₀₁, β₀₂ . . . and ΔTₘ by the user.

38. A focused ultrasonic therapeutic apparatus according to claim 36, wherein the signal processing and analyzing means automatically generates a plurality of data sets of β₀₁, β₀₂ . . . and ΔTₘ.

39. A focused ultrasonic therapeutic apparatus that can measure the temperature, comprising:
a high-energy focused ultrasonic source used for generating high-energy focused ultrasound to a particular region of human body to change the temperature thereof;
a positioning system, which is used for moving the particular region of human body to the focus of the high energy ultrasound, including a positioning B-type ultrasonic probe for imaging the particular region of the human body;
characterized in that,
the positioning B-type ultrasonic probe is in the B/M mode, transmitting a first ultrasonic wave to the particular region along the direction indicated with M-type ultrasound before the temperature of the region is changed, and then receiving a first echo wave from said first ultrasonic wave reflected by the human tissue in the target region and the tissue away from the target region; and transmitting a second ultrasonic wave to the particular region after the temperature of the region is changed, and then receiving a second echo wave from said second ultrasonic wave reflected by the human tissue in the target region and the tissue away from the target region so as to obtain a first echo wave parameter and a second echo wave parameter respectively;
a signal processing and analyzing means which extracts the temperature changing information of the particular region from the first wave parameter and the second echo wave parameter
wherein the signal processing and analyzing means calculates a theoretical comparison value between the first and second echo wave parameter, and optimizes the differences between the theoretical comparison values and the measured comparison values between the first and second echo wave parameters, such that the information of the local temperature changes of the particular region can be obtained through inversion.

40. A focused ultrasonic therapeutic apparatus according to claim 39, wherein said first echo parameter and said second echo parameter are sound pressure of the echo wave or power of the echo wave.

41. A focused ultrasonic therapeutic apparatus according to claim 39, wherein the formula used by the signal processing and analyzing means for calculating the theoretical comparison value between the second echo wave parameter and the first echo wave parameter is
\overline{p}=\overline{p}₀S(β₁,R₀)S(β₂,L) (11)
wherein the following empirical formulas are used:
$$S ( β , X ) = 1 - \frac{β {X}^{3}}{f} \qquad ( 15 )$$
βⱼ=β₀ⱼΔTₘg(f,ΔTₘ), (16)
\overline{p}₀=VA₀e^{ik(L+R}^{₀}⁾ (17)
\overline{p}₀ is sound pressure of the echo wave when there is no temperature field; \overline{p} is sound pressure of the echo wave when there is a temperature field; f is the frequency of the sound wave; g is a value to be determined; L and R₀ represent the distance from the transducer and the reflection plane to the center of the hot source respectively; ΔTₘ is the maximum temperature increase at the center of hot source relevant to ambient temperature, and the comparison value between the first echo wave parameter and the second echo wave parameter is defined as
$${I}_{1} ( {β}_{01} , {β}_{02} , … , Δ {T}_{m} , f ) = {( \frac{\overline{p}}{{\overline{p}}_{0}} )}^{2} \qquad ( {12}^{′} )$$
in which β₀₁, β₀₂ . . . are acoustic-thermal coupling parameters.

42. A focused ultrasonic therapeutic apparatus according to claim 41, wherein the acoustic-thermal coupling parameter is expressed as
$${β}_{0 j} = {\sum }_{i = 0}^{M} {α}_{ij} ( T ) {( Δ T )}^{i} \qquad ( 18 )$$

43. A focused ultrasonic therapeutic apparatus according to claim 42, wherein the acoustic-thermal coupling parameter is further expressed as
β₀ⱼ=β₀ⱼ⁽⁰⁾(ΔTₘ)[1+Δ] (19)
in which Δ is a specified fine variance.

44. A focused ultrasonic therapeutic apparatus according to claim 39, said signal processing and analyzing means performs fast Fourier transformation (FFT) and spectrum smoothing to the measured first echo wave parameter and second echo wave parameter, and obtains the minimum difference between the theoretical comparison values and the measured comparison values by means of the least square method so as to obtain local temperature increment of the particular region by means of inversion.

45. A focused ultrasonic therapeutic apparatus according to claim 44, wherein the signal processing and analyzing means obtains the temperature increment in the region to be measured by means of the inversion method, which can be expressed with the following formulas:
the sound pressure spectrum of the first echo wave parameter and the second echo wave parameter in the frequency domain are respectively p₀(fᵢ) and p₁(fᵢ), I₀(fᵢ) is defined as
$${I}_{0} ( {f}_{i} ) = {[ \frac{{p}_{1} ( {f}_{i} )}{{p}_{0} ( {f}_{i} )} ]}^{2} \qquad ( 13 )$$
i=1, . . . , N, N is the number the frequencies selected,
An objective function is defined as
$$Q = {\sum }_{i = 1}^{N} {\{ {I}_{0} ( {f}_{i} ) - {I}_{1} ( {β}_{01} , {β}_{02} , … , Δ {T}_{m} , {f}_{i} ) \}}^{2} \qquad ( 14 )$$
selecting β₁, β₂ . . . and ΔTₘ to make Q as the minimum value, the corresponding ΔTₘ is the differential value between the temperature of the hot source and the ambient temperature T₀.

46. A focused ultrasonic therapeutic apparatus according to claim 45, wherein the signal processing and analyzing means comprises an input means used for inputting a plurality of data sets of β₀₁, β₀₂ . . . and ΔTₘ by the user.

47. A focused ultrasonic therapeutic apparatus according to claim 45, wherein the signal processing and analyzing means automatically generates a plurality of data sets of β₀₁, β₀₂ . . . and ΔTₘ.
